(19) United States
(12) Patent Application Publication
(10) Pub. No.: US 2026/0002893 A1
(43) Pub. Date: Jan. 1, 2026

(54) SYSTEMS AND METHODS FOR MEASUREMENT OF FLUID COMPOSITION

(71) Applicant: Schlumberger Technology Corporation, Sugar Land, TX (US)

(72) Inventors: Massimiliano Fiore, Singapore (SG); Cheng-Gang Xie, Singapore (SG)

(21) Appl. No.: 18/754,247

(22) Filed: Jun. 26, 2024

(51) Int. Cl. G01N 23/083; G01N 33/28

(52) U.S. Cl. CPC G01N 23/083; G01N 33/2823

(57) ABSTRACT

A device may include a choke body having a first longitudinal end and a second longitudinal end with a choke volume therein from the first longitudinal end to the second longitudinal end, the choke volume having a center cross-sectional area of a center portion that is no more than a first end cross-sectional area. A device may include a wall of the choke body that continuous in a circumferential direction and having a first radiotransparency at a first end portion and having a second radiotransparency less than the first radiotransparency in the center portion proximate to the center cross-sectional area.

### BACKGROUND

Measurement of multiphase production fluids and/or formation fluids can be done in line at a production well. The accuracy of the measurements can vary by the flowrate of the fluid through the flow meter. Different flow meters are configured for different flow rate. As a well matures, the flow rate decreases, which impairs the accuracy of compositional measurements.

### SUMMARY

In some aspects, the techniques described herein relate to a device for measuring a composition, the device including: a choke body having a first longitudinal end and a second longitudinal end with a choke cross-sectional area therein from the first longitudinal end to the second longitudinal end, the choke cross-sectional area having a center cross-sectional area of a center portion that is no more than a first end cross-sectional area; and a wall of the choke body that continuous in a circumferential direction and having a first radiotransparency at a first end portion and having a second radiotransparency less than the first radiotransparency in the center portion proximate to the choke cross-sectional area.

In some aspects, the techniques described herein relate to a system for measuring a composition, the system including: a Venturi throat; a choke positioned in the Venturi throat wherein the choke reduces a sampling cross-sectional area of the Venturi throat to a choke cross-sectional area by a choke ratio of no more than 0.4; a broad-spectrum energy source configured to provide at least a high-energy photon and a low-energy photon to the sampling cross-sectional area; and a radiation detector located proximate the Venturi throat and opposite from the energy source.

In some aspects, the techniques described herein relate to a method of measuring a composition, the method including: providing a multiphase flow meter (MPFM) having a choke positioned in a sampling volume of the MPFM; flowing a formation fluid through the MPFM, wherein the formation fluid flows through the choke; emitting at least high-energy photons and very low-energy photons from an energy source through a center portion of the choke, wherein at least a portion of the high-energy photons and very low-energy photons pass through choke material in a sampling direction; and detecting the portion of the high-energy photons and very low-energy photons after passing through the choke at a detector.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and aspects of embodiments of the disclosure will be set forth in the description which follows, and in part will be obvious from the description, or may be learned by the practice of such embodiments. The features and aspects of such embodiments may be realized and obtained by means of the instruments and combinations particularly pointed out in the appended claims. These and other features will become more fully apparent from the following description and appended claims or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, non-schematic drawings should be considered as being to scale for some embodiments of the present disclosure, but not to scale for other embodiments contemplated herein. Understanding that the drawings depict some example embodiments, the embodiments will be described and explained with additional specificity and detail through the use of the accompanying drawings in which:

FIG. 1 is a schematic representation of a production well, according to at least some embodiments of the present disclosure.

FIG. 2-1 and FIG. 2-2 are cross-sectional view of a multiphase flow meter, according to at least some embodiments of the present disclosure.

FIG. 3 is a perspective cross-sectional view of an embodiment of a choke, according to at least some embodiments of the present disclosure.

FIG. 4 is a side cross-sectional view of choke including an insert, according to at least some embodiments of the present disclosure.

FIG. 5-1 through FIG. 5-6 are transverse cross-sectional views through a center portion of a choke, according to at least some embodiments of the present disclosure.

FIG. 6 is a partially cross-sectional view of an embodiment of an energy source used in an MPFM, according to at least some embodiments of the present disclosure.

FIG. 7 shows the attenuation triangle with photon emissions from a broad-spectrum energy source, according to at least some embodiments of the present disclosure.

FIG. 8 illustrates experimental data for gas hold-ups (GHU) with an XRF energy source, according to at least some embodiments of the present disclosure.

### DETAILED DESCRIPTION

Embodiments of the present disclosure generally relate to devices, systems, and methods for the measurement of hydrocarbons produced by a well. Revenue from oil and gas wells is based, among other factors, on the declared hydrocarbon production of the well. Precise and accurate measurements of the volume and mass of the hydrocarbons produced from a well and/or wellhead is needed to correctly compensate the well owner and/or operator. In some examples, a well or wellhead has a large variation in production over the lifetime of the well. For example, unconventional oil and gas development has become the most widespread form of energy production in the United States and in other regions of the world. A multiphase flow meter (MPFM) can measure a plurality of different products exiting a well and quantify the production of the well in real-time without the need for separators.

A challenge to ensure accuracy throughout the life of unconventional wells is a rapid and irreversible production decline, on average, halved after the first year. In such a production scenario, the differential pressure (dP) across the throat of the MPFM may become so low that flow rate accuracy can deteriorate. In addition to unconventional wells, this same challenge exists with aging conventional wells at a lesser rate.

In some embodiments, devices, systems, and methods according to the present disclosure allow a single MPFM to operate reliably, accurately, and precisely across an increased range of liquid flowrates of a well down to and including less than 50 barrels per day. In some embodiments, a choke is inserted into a throat of the MPFM to reduce a cross-sectional area of the sampling region of the MPFM to create an equivalent or similar flowrate through the sampling region. In some embodiments, an energy source for the MPFM produces a broad-spectrum input energy that includes at least high-energy photons (i.e., gamma radiation) and low-energy photons (i.e., low-energy x-ray radiation) to measure the phase fractions (i.e., the volumetric composition of the three phases in the multiphase flow) and reduce measurement uncertainty in low flowrates. In some embodiments, the choke reduces the sampling volume proximate the energy source and a detector of the MPFM to a choke cross-sectional area by a choke ratio that is no more than 0.4 (e.g., the choke cross-sectional area is no more than 40% of the original cross-sectional area of the sampling volume of a throat of the MPFM).

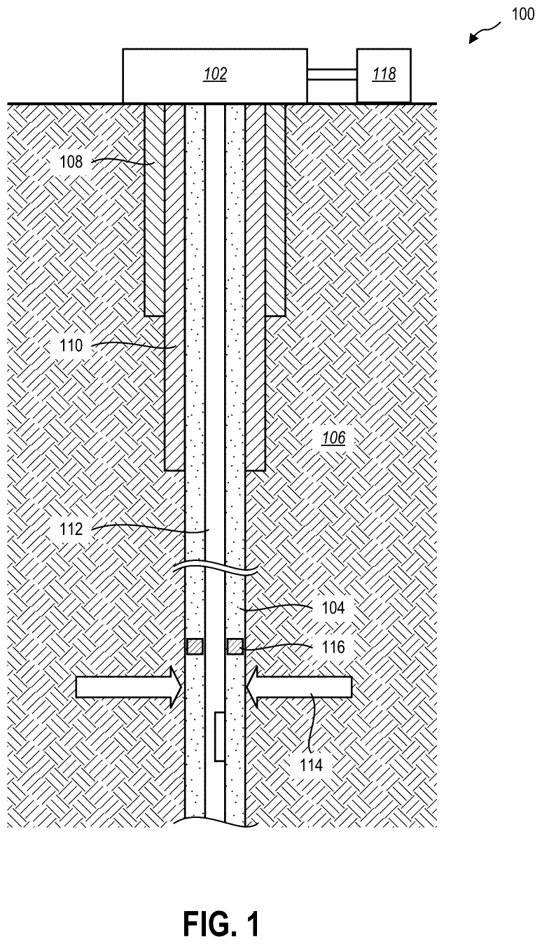

FIG. 1 is a schematic representation of a production well 100. In some embodiments, the production well 100 includes a wellhead 102 that caps a wellbore 104. The wellbore 104 is located in a formation 106 containing fluid hydrocarbon reserves. For example, the formation 106 may include liquid hydrocarbons, gaseous hydrocarbon, liquid hydrocarbons that expand into a gas upon release of subsurface pressure, and combinations thereof. In some embodiments, the wellbore 104 is supported by one or more casings, such as a surface casing 108 and/or an intermediate casing 110. In some embodiments, at least a portion of the wellbore 104 is uncased.

The wellhead 102 is connected to a pipe 112, such as a production pipe, which receives formation fluid 114 from the formation 106 in a downhole environment and conveys the formation fluid 114 upward through the pipe 112 to the wellhead 102 at the surface. In some embodiments, the production location in the wellbore 104 is isolated or partially isolated by one or more packers 116 expanded between the pipe 112 and a wall of the wellbore 104 (whether that wall is casing or directly adjacent to the formation 106).

In some embodiments, at least a portion of the formation fluid 114 conveyed to the surface by the pipe 112 is provided to one or more sensors to measure the amount and proportion of hydrocarbons in the formation fluid 114. In some embodiments, at least a portion of the formation fluid 114 is provided to an MPFM 118 for measurement and analysis at or near the wellhead 102 of the well 100.

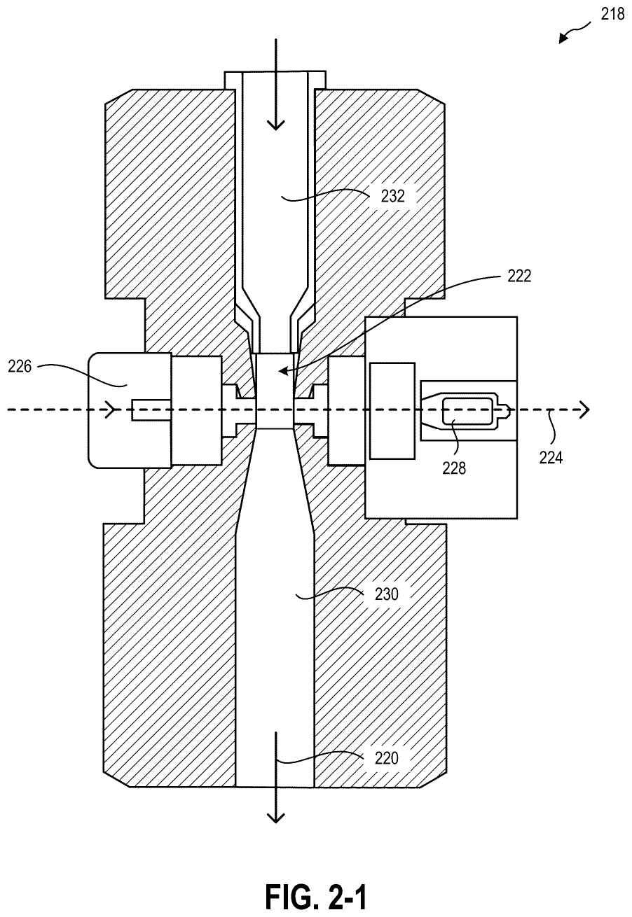

FIG. 2-1 and FIG. 2-2 illustrate an embodiment of an MPFM 218 according to the present disclosure. FIG. 2-1 is a side cross-sectional view of the embodiment of an MPFM 218 parallel to a flow direction of formation fluid through the MPFM 218. FIG. 2-2 is a top view of the embodiment of an MPFM 218 transverse to the flow direction 220. The MPFM 218 includes a Venturi throat forming a sampling volume 222 through which a signal is provided in a sampling direction 224. In some embodiments, the sampling direction 224 is perpendicular to the flow direction 220.

The MPFM 218 includes a radioactive source 226 that emits photons in the sampling direction 224 through the sampling volume 222. In some embodiments, a detector 228 (such as a scintillation detector) located opposite from the radioactive source across the sampling volume 222 receives the transmitted photons from the radioactive source 226 and counts the transmitted photons. The composition of the formation fluid in the sampling volume 222 changes the detected photons at the detector 228. The determination of the formation fluid composition and the resulting phase fraction of hydrocarbons and water present in the formation fluid is based at least partially on an empty pipe measurement of the sampling volume 222, a known flowrate through the sampling volume 222, a sampling path length (d) of the formation fluid through the sampling volume 222, density of the formation fluid, and detected count rates while flowing formation fluid through the sampling volume 222.

In some embodiments, the MPFM 218 is configured to receive a formation fluid through the Venturi throat 230 and through sampling volume 222. The Venturi throat and sampling path length (d) of the MPFM 218 is configured for a range of fluid flowrates through the sampling volume 222. When the production of the well matures, and the flowrate changes, the accuracy of the compositional measurements can degrade.

To avoid replacing an MPFM with a smaller one as the well matures (for example, to limit capital expenditures and nonproductive time), the installation of a choke 232 reduces the cross-sectional area of the sampling volume 222 and increases the measured dP for low flow rates through the sampling volume 222. The increased turndown ratio and a dedicated flow model for an MPFM 218 with a choke 232 installed, in some embodiments, increases the range of flowrates for which the MPFM 218 may accurately operate, increasing the operational lifetime.

In some embodiments, the choke 232 in the sampling volume 222 reduces the cross-sectional area while keeping the same sampling path length (d) between the photons of the energy source 226 and the formation fluid in the sampling volume 222.

However, reducing the cross-sectional area at the MPFM sampling volume 222 of the Venturi throat 230 by impinging in a direction transverse to the sampling direction 224 is limited by the increasingly narrow choke cross-sectional area defined by the choke 232. For example, properly modeling the formation fluid flowing through such a high aspect-ratio region becomes virtually impossible. In some embodiments, a choke according to the present disclosure reduces the size of the sampling volume 222 to a choke cross-sectional area based on a choke ratio no more than 0.4 while maintaining accuracy in the interaction calculations.

The X- and γ-ray emission after radioactive decay is a random process well described by the Poisson distribution. The count rate n, measured without of loss of generality in 1/s and defined as number of measured X- and γ-rays n transmitted by a radioactive energy source (such as radioactive source 226 described in relation to FIGS. 2-1 and 2-2) per second and acquired over the time t (in s), is affected by the uncertainty according to:

$${σ}_{n} = \sqrt{( n / t )}$$

This measurement uncertainty (On) propagates according to the linear attenuation coefficients, phase fractions, and, ultimately, to the flow rates. In general, by solely propagating the count rate uncertainty in flowing conditions of the above relationship, the uncertainty of linear attenuation coefficients/from the Beer-Lambert law n=n₀ exp(−λ·d) reads

$${σ}_{λ} = \frac{1}{d} \sqrt{\frac{1}{n \cdot t}}$$

where n₀ is the count rate when no flowing mixture interacts along the length of the radioactive source emissions, e.g., the sampling path length (d). By propagating the uncertainty of the above relationship, it can be demonstrated that the uncertainties of the phase fractions αᵢ (i stands for either “oil”, o, “gas”, g, or water. w) and uncertainties of the water-liquid ratio

$$W L R = \frac{{α}_{w}}{{α}_{w} + {α}_{o}}$$

read

$${σ}_{{α}_{g}} ∝ \frac{1}{d} \frac{1}{\sqrt{t}} ,$$

$${σ}_{WLR} ∝ \frac{1}{d} \frac{1}{( 1 - {α}_{g} ) \cdot \sqrt{t}}$$

for the gas phase fraction, affecting the liquid flow rate uncertainty, and the WLR, affecting both oil and water flow rate uncertainties from the liquid flow rate, respectively. The proportionality to 1/d of the WLR and gas fraction uncertainties demonstrate that the smaller the sampling path length (d), the greater the uncertainty in phase fraction calculations.

A conventional choke design maintains the same interaction path length for the energy source emissions (e.g., photons) with the formation fluid flow. For smaller and smaller well production rates, the choke cross-sectional area through the choke 232 may become too narrow.

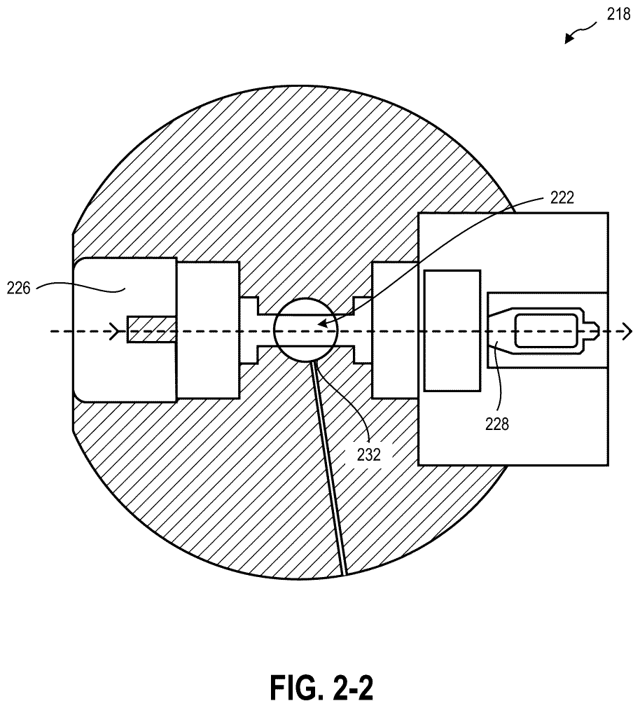

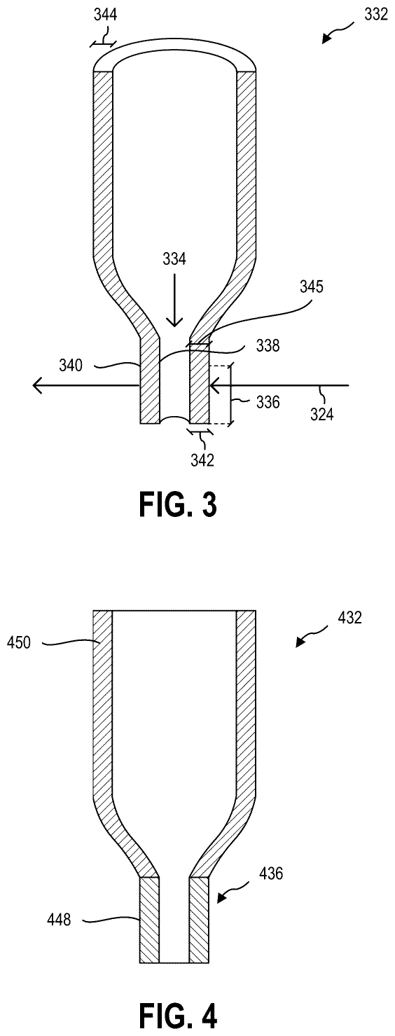

FIG. 3 is a perspective cross-sectional view of an embodiment of an additional choke 332 according to the present disclosure. In some embodiments, the choke 232 described in relation to FIG. 2 and other embodiments of chokes according to the present disclosure may extend the operational lifetime of an MPFM by providing different choke cross-sectional areas for the same MPFM. For example, a kit may be provide including an MPFM and a plurality of chokes having different choke cross-sectional areas according to some embodiments of the present disclosure.

In some embodiments, the choke 332 has a choke ratio of no more than 0.4. In some embodiments, the choke ratio is defined by the choke cross-sectional area 334 (e.g., on the inside of the center portion 336 of the choke 332) relative to the sampling volume cross-sectional area of the Venturi throat (such as the Venturi throat 230 of FIG. 2-1 and FIG. 2-2) proximate to the sampling direction 324. In some embodiments, such as embodiments with a circular choke cross-sectional area, as will be described in more detail herein, the choke ratio is a circular area defined by an inner surface 338 of the choke 332 proximate to the sampling direction 324 relative to a circular area defined by an outer surface 340 of the choke 332 proximate to the sampling direction 324.

In some embodiments, the choke 332 has a wall that is substantially continuous in a circumferential direction and has a sampling wall thickness 342 proximate to the center portion 336 of the choke 332 and a first end wall thickness 344 distal from the center portion 336 of the choke 332. The first end thickness 344 may be thicker than the sampling wall thickness 342 to provide support to the choke 332 and/or provide a more erosion-resistant choke 332 as the formation fluid flows from the first end 346 toward the center portion 336. In some embodiments, the wall thickness varies continuously between the first end 346 and the center portion 336 with a transition wall thickness 345 between the sampling wall thickness 342 and the first end thickness 344. In some embodiments, the wall thickness varies discontinuously, such as with an abrupt change in thickness in a longitudinal location between the first end 346 and the center portion 336.

In some embodiments, the different wall thicknesses between the sampling wall thickness 342 and the first end thickness 344 creates a difference in radiotransparency (relative to the photons emitted by the radioactive source in the sampling direction 324) between the center portion 336 and the first end 346. In some embodiments, the choke 332 has a first radiotransparency proximate to a first end 346 of the choke 332, and a second radiotransparency that is less than the first radiotransparency proximate to the center portion 336 of the choke 332.

In some embodiments, the material of the choke 332 is selected to be a low-absorbing material, such as a low-Z material that absorbs or otherwise interacts with the photons emitted by the energy source less than the metal of a conventional choke such that the photons pass through the low-absorbing choke material with little to no interference. For example, the X- and γ-ray absorption may be minimized by selecting a low-absorbing material for the center portion 336 of the choke 332 in the γ-ray path, such as plastic or similar. In at least one example, the low-absorbing material is polyether ether ketone (PEEK). In some embodiments, the entire choke 332 is a monolithic piece of material, such as a monolithic piece of PEEK. In some embodiments, the choke 332 includes a plurality of different materials, such as a low-absorbing material proximate to the center portion 336 and a different material proximate to the first end 346.

In some embodiments, the low-Z material may be located in an insert 448 at the center portion 436, such as illustrated in the side cross-sectional view of FIG. 4. The upper body 450 of the choke 432 includes a second material with a radiotransparency different from the radiotransparency of the low-absorbing material of the insert 448. For example, the upper body 450 may be a metal that provides greater erosion resistance to the formation fluid flowing through the choke 432 than PEEK used to form the insert 448. The insert 448 has a greater radiotransparency (i.e., lower photon absorption) than the metal of the upper body 450. In some embodiments, the wall thicknesses of the center portion 436 and/or the upper body 450 may vary, such as described in relation to FIG. 3, in addition to material changes between the upper body 450 and the insert 448 proximate to the center portion 436.

As described herein, some embodiments of chokes according to the present disclosure have a radiotransparency that changes from the first end toward the center portion. In some embodiments, a choke has a center portion that varies in radiotransparency in radial directions relative to the sampling direction. For example, the choke may have a center portion that varies in radiotransparency relative to different radial directions.

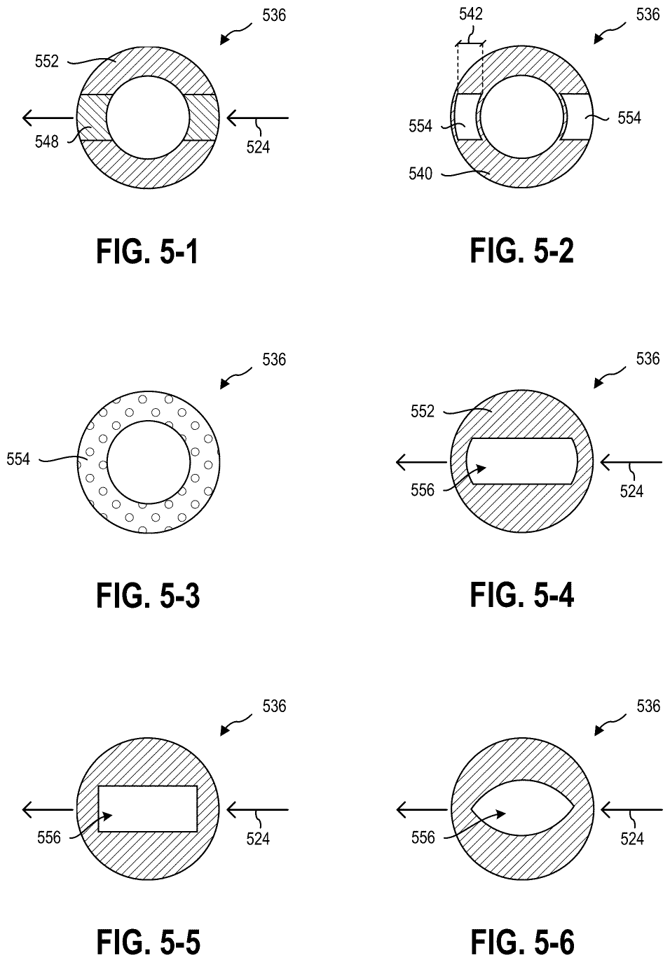

FIG. 5-1 is a transverse cross-sectional view through an embodiment of a center portion 536 of a choke. In some embodiments, the center portion 536 includes an insert 548 in the center portion 536 that is less than the entire circumference of the center portion 536. For example, the insert(s) 548 may be positioned in the radial direction of the center portion 536 in the sampling direction 524. In some embodiments, the inserts 548 are positioned in line with the sampling direction 524 to increase the radiotransparency of the choke in the center portion 536, while the lateral sides 552 include a second material that has a lower radiotransparency. In some embodiments, the second material has a greater hardness, greater erosion resistance, greater toughness, or other materials that allow the lateral sides 552 to provide support to the inserts 548. In at least one embodiment, the material of the lateral sides 552 is integrally formed with an upper body of the choke, such as the upper body described in relation to FIG. 4. In some embodiments, the inserts 548 include PEEK or another low-absorbing material.

The radiotransparency in the sampling direction may be increased by other designs, which may be used in combination with any designs described herein. FIG. 5-2 is a transverse cross-sectional view through another embodiment of a center portion 536 of a choke. In some embodiments, the center portion 536 includes one or more voids 554 within the sampling wall thickness 542. In some embodiments, the void 554 is a recess in the outer surface 540 of the center portion 536. In some embodiments, the void 554 is entirely within the sampling wall thickness 542 and the outer surface 540 is substantially continuous across and/or around the void 554.

FIG. 5-3 is a transverse cross-sectional view through another embodiment of a center portion 536 of a choke. In some embodiments, at least a portion of the center portion 536 includes a material that is porous. For example, the low-absorbing material may be a porous material with a plurality of voids 554 therein that reduces the interaction between material of wall(s) of the center portion 536 and the photons from energy source before the detector.

FIG. 5-4 is a transverse cross-sectional view through another embodiment of a center portion 536 of a choke. In some embodiments, the choke cross-sectional area 556 through which the formation fluid flows within the choke is circular, such as illustrated in embodiments of FIG. 5-1 through FIG. 5-3. In some embodiments, the choke cross-sectional area 556 is non-circular. In some embodiments, the choke cross-sectional area 556 is elongated in the sampling direction 524, and the sampling wall thickness varies such that less material of the center portion 536 is present in the sampling direction 524 to interact with the photons. For example, FIG. 5-4 illustrates an embodiment of a center portion 536 with a choke cross-sectional area 556 that is a truncated circle. In other words, the ends of the choke cross-sectional area 556 aligned in the sampling direction 524 have a substantially circular curvature (i.e., arc segment of a circular choke cross-sectional area 556) while the edges adjacent to the lateral sides 552 are substantially parallel to the sampling direction 524. A hybrid shape of the insert between the circular and the conventional choke profile may reduce or limit the interaction with the X- and γ-ray photons while maintaining a longer sampling path length through the choke cross-sectional area 556 when compared with a circular choke cross-sectional area 556. In some embodiments, the sampling wall thickness at either end of the choke cross-sectional area 556 in the sampling direction 524 is less than the wall thickness on the lateral side(s) 552.

FIG. 5-5 is a transverse cross-sectional view through another embodiment of a center portion 536 of a choke. It should be understood that while each choke cross-sectional area 556 has been described herein as circular or a portion of a circle, other shapes are considered. In some embodiment, the choke cross-sectional area 556 is rectilinear, such as a rectangle. For example, a rectangular choke cross-sectional area 556 may be aligned with a long axis in the sampling direction.

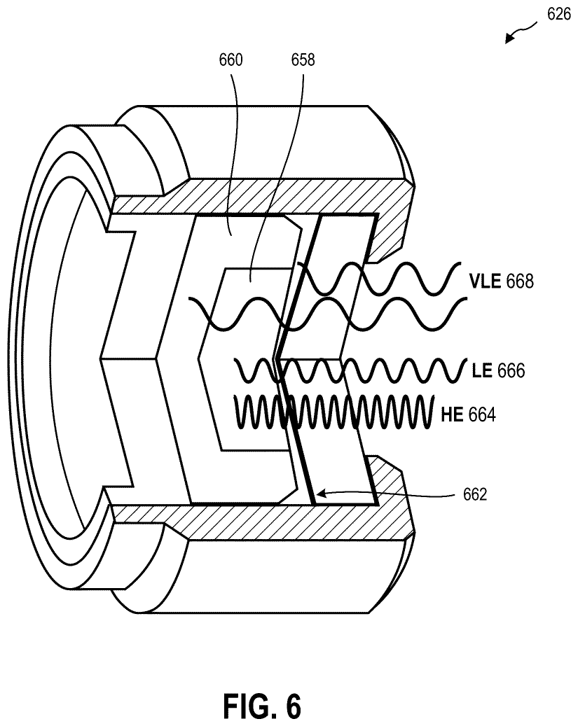

FIG. 5-6 is a transverse cross-sectional view through another embodiment of a center portion 536 of a choke. In some embodiments, the choke cross-sectional area 556 is elliptical with a major axis of the choke cross-sectional area 556 substantially aligned with the sampling direction 524. In some embodiments, the major axis of the choke cross-sectional area 556 is no less than 50% of a throat major axis of the center cross-sectional area of the throat. For example, the major axis of the choke cross-sectional area 556 is no less than 50% of an outer diameter of the center portion 536.

FIG. 6 is a partially cross-sectional view of an embodiment of a radioactive source 626 used in an MPFM according to the present disclosure. In some embodiments, the radioactive source 626 is an X-ray fluorescence (XRF) energy source that emits at least high-energy photons and low-energy photons. In some embodiments, the XRF energy source emits high-energy photons (i.e., γ-ray), low-energy photons (i.e., high energy X-ray), and very low-energy photons (i.e., lower energy X-ray). In some embodiments, the XRF energy source 626 includes a radioactive source 658 that energizes a fluorescence source 660 and/or a fluorescence foil 662 adjacent to and/or around the radioactive source 658.

In some embodiments, the radioactive source 658 produces high-energy photons 668 and low-energy photons 666. At least some of the high-energy photons 668 and/or low-energy photons 666 impart energy to the fluorescence source 660 and/or the fluorescence foil 662 to cause fluorescence and emission of very low-energy photons 668 from the fluorescence source 660 and/or the fluorescence foil 662. The broad-spectrum emission from the energy source 626 may further improve the accuracy of an embodiment of an MPFM.

In some embodiments, the fluorescence source 660 is or includes a Ba-133-based ceramic matrix to emit naturally photons having energy levels of approximately 32 keV (e.g., low-energy photons 666) and 81 keV (e.g., high-energy photons 668). In some embodiments, the 32 keV energy level is made of or includes a plurality of energy levels, such as 31 keV and 35 keV X-rays naturally emitted by the Ba-133 radioisotope. In some embodiments, the very low-energy photons 668 are generated primarily by the interaction of part of the 32-keV photons (low-energy photons 666), and the very low-energy photons 668 have an energy less than that of the low-energy photons 666. For example, the very low-energy photons 668 may have an energy between 15 keV and 25 keV, depending on the material of the fluorescence foil 662.

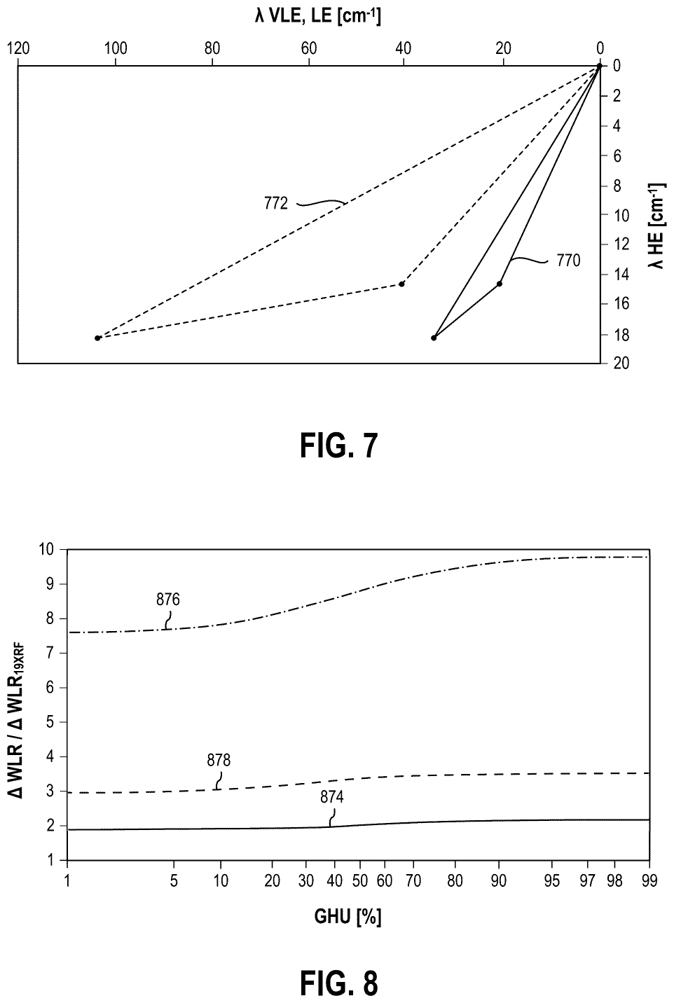

The uncertainty reduction from the use of the XRF source can be graphically illustrated by the typical representation for MPFMs based on multi-energy γ-ray and x-ray absorption of the so-called attenuation triangle. FIG. 7 shows the comparison of the attenuation triangle with the emissions from a Ba-133 source. The natural emission triangle 770 is from the natural emissions LE and HE, whereas the XRF triangle 772 is from the VLE and HE emissions from an XRF Ba-133 source. The larger difference in linear attenuation coefficients for an XRF source leads to a wider triangle, indication of an uncertainty reduction in differentiating oil and water in multiphase conditions. It is important to point out that, for the same fluids, the attenuation triangle is related to the energy of the incident photons: for the same flow conditions, the increased WLR uncertainty when reducing the length of the interaction path will be decreased by employing the fluorescence X-rays from an XRF energy source, such as described in relation to FIG. 6. In addition to reducing the WLR uncertainty, in some embodiments, longer wavelength photons (e.g., very low-energy photons) interact with the low-Z material or other radiotransparent materials less, further improving the sampling interactions.

FIG. 8 illustrates experimental data for gas hold-ups (GHU) with an XRF energy source, according to at least some embodiments of the present disclosure. FIG. 8 shows the theoretical WLR uncertainty, solely due to the Poisson noise, relative to the theoretical AWLR of an MPFM having a 19 mm diameter sampling volume and an XRF energy source. A first trendline 874 illustrates the WLR uncertainties for an MPFM having a 19 mm diameter sampling volume with a 10 mm diameter circular-shaped choke made of PEEK are expected to be only doubled for all GHUs. For the same MPFM and choke but using a standard energy source emitting only high-energy and low-energy photons (i.e., no very low-energy photons of the XRF energy source), a second trendline 876 illustrates the WLR uncertainties amplified up to 10 times for high GHUs. The third trendline 878 illustrates the approximately 3-3.5 times increase in AWLR using the standard energy source without the choke shortening the sampling path length. The results, therefore, indicate a synergistic advantage of using an XRF energy source in combination with chokes having a short interaction path length. In each example, a choke having a cross-sectional area greater than the illustrated 10 mm diameter may further improve the AWLR measurements.

### INDUSTRIAL APPLICABILITY

Embodiments of the present disclosure generally relate to devices, systems, and methods for the measurement of hydrocarbons produced by a well. Revenue from oil and gas wells is based, among other factors, on the declared hydrocarbon production of the well. Precise and accurate measurements of the volume and mass of the hydrocarbons produced from a well and/or wellhead is needed to correctly compensate the well owner and/or operator. In some examples, a well or wellhead has a large variation in production over the lifetime of the well. For example, unconventional oil and gas development has become the most widespread form of energy production in the United States and in other regions of the world. A multiphase flow meter (MPFM) can measure a plurality of different products exiting a well and quantify the production of the well in real-time without the need for separators.

A challenge to ensure accuracy throughout the life of unconventional wells is a rapid and irreversible production decline, on average, halved after the first year. In such a production scenario, the differential pressure (dP) across the throat of the MPFM may become so low that flow rate accuracy can deteriorate. In addition to unconventional wells, this same challenge exists with aging conventional wells at a lesser rate.

In some embodiments, devices, systems, and methods according to the present disclosure allow a single MPFM to operate reliably, accurately, and precisely across an increased range of liquid flowrates of a well down to and including less than 50 barrels per day. In some embodiments, a choke is inserted into a throat of the MPFM to reduce a cross-sectional area of the sampling region of the MPFM to create an equivalent or similar flowrate through the sampling region. In some embodiments, an energy source for the MPFM produces a broad-spectrum input energy that includes at least high-energy photons (i.e., gamma radiation) and low-energy photons (i.e., low-energy x-ray radiation) to measure the phase fractions (i.e., the volumetric composition of the three phases in the multiphase flow) and reduce measurement uncertainty in low flowrates. In some embodiments, the choke reduces the sampling volume proximate the energy source and a detector of the MPFM to a choke cross-sectional area by a choke ratio that is no more than 0.4 (e.g., the choke cross-sectional area is no more than 40% of the original cross-sectional area of the sampling volume of a throat of the MPFM).

In some embodiments, an MPFM includes a Venturi throat through which a signal is provided in a sampling direction. In some embodiments, the sampling direction is perpendicular to the flow direction. The MPFM includes a radioactive source that emits photons in the sampling direction through the sampling volume of the Venturi throat. In some embodiments, a detector (such as a scintillation detector) located opposite from the radioactive source across the sampling volume of the Venturi throat receives the transmitted photons from the radioactive source and counts the transmitted photons. The composition of the formation fluid in the sampling volume changes the detected photons at the detector. The determination of the formation fluid composition and the resulting phase fraction of hydrocarbons and water present in the formation fluid is based at least partially on an empty pipe measurement of the sampling volume, a known flowrate through the Venturi throat, a sampling path length (d) of the formation fluid through the Venturi throat, density of the formation fluid, and detected count rates while flowing formation fluid through the Venturi throat.

In some embodiments, the MPFM is configured to receive a formation fluid through the Venturi throat and sampling volume. The Venturi throat and sampling path length (d) of the MPFM is configured for a range of fluid flowrates through the sampling volume of the Venturi throat. When the production of the well matures, and the flowrate changes, the accuracy of the compositional measurements can degrade.

To avoid replacing an MPFM with a smaller one as the well matures (for example, to limit capital expenditures and nonproductive time), the installation of a choke reduces the cross-sectional area of the Venturi throat and increases the measured dP for low flow rates through the Venturi throat. The increased turndown ratio and a dedicated flow model for an MPFM with a choke installed, in some embodiments, increases the range of flowrates for which the MPFM may accurately operate, increasing the operational lifetime.

In some embodiments, the choke in the sampling volume of the Venturi throat reduces the cross-sectional area while keeping the same sampling path length (d) between the photons of the energy source and the formation fluid in the sampling volume.

However, reducing the cross-sectional area at the MPFM Venturi throat by impinging in a direction transverse to the sampling direction is limited by the increasingly narrow choke cross-sectional area defined by the choke. For example, properly modeling the formation fluid flowing through such a high aspect-ratio region becomes virtually impossible. In some embodiments, a choke according to the present disclosure reduces the size of the sampling volume of the Venturi throat to a choke cross-sectional area based on a choke ratio no more than 0.4 while maintaining accuracy in the interaction calculations.

The X- and γ-ray emission after radioactive decay is a random process well described by the Poisson distribution. The count rate n, measured without of loss of generality in 1/s and defined as number of measured X- and γ-rays n transmitted by a radioactive energy source per second and acquired over the time t (in s), is affected by the uncertainty according to:

$${σ}_{n} = \sqrt{( n / t )}$$

This measurement uncertainty (σₙ) propagates according to the linear attenuation coefficients, phase fractions, and, ultimately, to the flow rates. In general, by solely propagating the count rate uncertainty in flowing conditions of the above relationship, the uncertainty of linear attenuation coefficients λ from the Beer-Lambert law n=n₀ exp (−λ·d) reads

$${σ}_{λ} = \frac{1}{d} \sqrt{\frac{1}{n \cdot t}}$$

where n₀ is the count rate when no flowing mixture interacts along the length of the radioactive source emissions, e.g., the sampling path length (d). By propagating the uncertainty of the above relationship, it can be demonstrated that the uncertainties of the phase fractions αᵢ (i stands for either “oil”, o, “gas”, g, or water. w) and uncertainties of the water-liquid ratio

$$W L R = \frac{{α}_{w}}{{α}_{w} + {α}_{o}}$$

read

$${σ}_{{α}_{g}} ∝ \frac{1}{d} \frac{1}{\sqrt{t}} ,$$

$${σ}_{WLR} ∝ \frac{1}{d} \frac{1}{( 1 - {α}_{g} ) \cdot \sqrt{t}}$$

for the gas phase fraction, affecting the liquid flow rate uncertainty, and the WLR, affecting both oil and water flow rate uncertainties from the liquid flow rate, respectively. The proportionality to 1/d of the WLR and gas fraction uncertainties demonstrate that the smaller the sampling path length (d), the greater the uncertainty in phase fraction calculations.

A conventional choke design maintains the same interaction path length for the energy source emissions (e.g., photons) with the formation fluid flow. For smaller and smaller well production rates, the choke cross-sectional area through the choke may become too narrow.

In some embodiments, chokes according to the present disclosure may extend the operational lifetime of an MPFM by providing different choke cross-sectional areas for the same MPFM. For example, a kit may be provide including an MPFM and a plurality of chokes having different choke cross-sectional areas according to some embodiments of the present disclosure.

In some embodiments, the choke has a choke ratio of no more than 0.4. In some embodiments, the choke ratio is defined by the choke cross-sectional area (e.g., on the inside of the center portion of the choke) relative to the sampling volume cross-sectional area proximate to the sampling direction. In some embodiments, such as embodiments with a circular choke cross-sectional area, as will be described in more detail herein, the choke ratio is a circular area defined by an inner surface of the choke proximate to the sampling direction relative to a circular area defined by an outer surface of the choke proximate to the sampling direction.

In some embodiments, the choke has a wall that is substantially continuous in a circumferential direction and has a sampling wall thickness proximate to the center portion of the choke and a first end wall thickness distal from the center portion of the choke. The first end thickness may be thicker than the sampling wall thickness to provide support to the choke and/or provide a more erosion-resistant choke as the formation fluid flows from the first end toward the center portion. In some embodiments, the wall thickness varies continuously between the first end and the center portion with a transition wall thickness between the sampling wall thickness and the first end thickness. In some embodiments, the wall thickness varies discontinuously, such as with an abrupt change in thickness in a longitudinal location between the first end and the center portion.

In some embodiments, the different wall thicknesses between the sampling wall thickness and the first end thickness creates a difference in radiotransparency (relative to the photons emitted by the radioactive source in the sampling direction) between the center portion and the first end. In some embodiments, the choke has a first radiotransparency proximate to a first end of the choke, and a second radiotransparency that is less than the first radiotransparency proximate to the center portion of the choke.

In some embodiments, the material of the choke is selected to be a low-absorbing material, such as a low-Z material that absorbs or otherwise interacts with the photons emitted by the energy source less than the metal of a conventional choke such that the photons pass through the low-absorbing choke material with little to no interference. For example, the X- and γ-ray absorption may be minimized by selecting a low-absorbing material for the center portion of the choke in the y-ray path, such as plastic or similar. In at least one example, the low-absorbing material is polyether ether ketone (PEEK). In some embodiments, the entire choke is a monolithic piece of material, such as a monolithic piece of PEEK. In some embodiments, the choke includes a plurality of different materials, such as a low-absorbing material proximate to the center portion and a different material proximate to the first end.

In some embodiments, the low-Z material may be located in an insert at the center portion. The upper body of the choke includes a second material with a radiotransparency different from the radiotransparency of the low-absorbing material of the insert. For example, the upper body may be a metal that provides greater erosion resistance to the formation fluid flowing through the choke than PEEK used to form the insert. The insert has a greater radiotransparency (i.e., lower photon absorption) than the metal of the upper body. In some embodiments, the wall thicknesses of the center portion and/or the upper body may vary, in addition to material changes between the upper body and the insert proximate to the center portion.

As described herein, some embodiments of chokes according to the present disclosure have a radiotransparency that changes from the first end toward the center portion. In some embodiments, a choke has a center portion that varies in radiotransparency in radial directions relative to the sampling direction. For example, the choke may have a center portion that varies in radiotransparency relative to different radial directions.

In some embodiments, the center portion includes an insert in the center portion that is less than the entire circumference of the center portion. For example, the insert(s) may be positioned in the radial direction of the center portion in the sampling direction. In some embodiments, the inserts are positioned in line with the sampling direction to increase the radiotransparency of the choke in the center portion, while the lateral sides include a second material that has a lower radiotransparency. In some embodiments, the second material has a greater hardness, greater erosion resistance, greater toughness, or other materials that allow the lateral sides to provide support to the inserts. In at least one embodiment, the material of the lateral sides is integrally formed with an upper body of the choke. In some embodiments, the inserts include PEEK or another low-absorbing material.

The radiotransparency in the sampling direction may be increased by other designs, which may be used in combination with any designs described herein. In some embodiments, the center portion includes one or more voids within the sampling wall thickness. In some embodiments, the void is a recess in the outer surface of the center portion. In some embodiments, the void is entirely within the sampling wall thickness and the outer surface is substantially continuous across and/or around the void.

In some embodiments, at least a portion of the center portion includes a material that is porous. For example, the low-absorbing material may be a porous material with a plurality of voids therein that reduces the interaction between material of wall(s) of the center portion and the photons from energy source before the detector.

In some embodiments, the choke cross-sectional area through which the formation fluid flows within the choke is circular. In some embodiments, the choke cross-sectional area is non-circular. In some embodiments, the choke cross-sectional area is elongated in the sampling direction, and the sampling wall thickness varies such that less material of the center portion is present in the sampling direction to interact with the photons. In some embodiments, a center portion has a choke cross-sectional area that is a truncated circle. In other words, the ends of the choke cross-sectional area aligned in the sampling direction have a substantially circular curvature (i.e., arc segment of a circular choke cross-sectional area) while the edges adjacent to the lateral sides are substantially parallel to the sampling direction. A hybrid shape of the insert between the circular and the conventional choke profile may reduce or limit the interaction with the X- and y-ray photons while maintaining a longer sampling path length through the choke cross-sectional area when compared with a circular choke cross-sectional area. In some embodiments, the sampling wall thickness at either end of the choke cross-sectional area in the sampling direction is less than the wall thickness on the lateral side(s).

It should be understood that while each choke cross-sectional area has been described herein as circular or a portion of a circle, other shapes are considered. In some embodiment, the choke cross-sectional area is rectilinear, such as a rectangle. For example, a rectangular choke cross-sectional area may be aligned with a long axis in the sampling direction.

In some embodiments, the choke cross-sectional area is elliptical with a major axis of the choke cross-sectional area substantially aligned with the sampling direction. In some embodiments, the major axis of the choke cross-sectional area is no less than 50% of a throat major axis of the center cross-sectional area of the throat. For example, the major axis of the choke cross-sectional area is no less than 50% of an outer diameter of the center portion.

In some embodiments, the radioactive source is an X-ray fluorescence (XRF) energy source that emits at least high-energy photons and low-energy photons. In some embodiments, the XRF energy source emits high-energy photons (i.e., γ-ray), low-energy photons (i.e., high energy X-ray), and very low-energy photons (i.e., lower energy X-ray). In some embodiments, the XRF energy source includes a radioactive source that energizes a fluorescence source and/or a fluorescence foil adjacent to and/or around the radioactive source.

In some embodiments, the radioactive source produces high-energy photons and low-energy photons. At least some of the high-energy photons and/or low-energy photons impart energy to the fluorescence source and/or the fluorescence foil to cause fluorescence and emission of very low-energy photons from the fluorescence source and/or the fluorescence foil. The broad-spectrum emission from the energy source may further improve the accuracy of an embodiment of an MPFM.

In some embodiments, the fluorescence source is or includes a Ba-133-based ceramic matrix to emit naturally photons having energy levels of approximately 32 keV (e.g., low-energy photons) and 81 keV (e.g., high-energy photons). In some embodiments, the 32 keV energy level is made of or includes a plurality of energy levels, such as 31 keV and 35 keV X-rays naturally emitted by the Ba-133 radioisotope. In some embodiments, the very low-energy photons are generated primarily by the interaction of part of the 32-keV photons (low-energy photons), and the very low-energy photons have an energy less than that of the low-energy photons. For example, the very low-energy photons may have an energy between 15 keV and 25 keV, depending on the material of the fluorescence foil.

The present disclosure relates to systems and methods for measuring a composition of a fluid according to any of the following:

Clause 1. A device for measuring a composition, the device comprising: a choke body having a first longitudinal end and a second longitudinal end with a choke volume therein from the first longitudinal end to the second longitudinal end, the choke volume having a center cross-sectional area of a center portion that is no more than a first end cross-sectional area; and a wall of the choke body that continuous in a circumferential direction and having a first radiotransparency at a first end portion and having a second radiotransparency less than the first radiotransparency in the center portion proximate to the center cross-sectional area.

Clause 2. The device of clause 1, wherein the first end portion and the center portion include different materials.

Clause 3. The device of clause 1 or 2, wherein the center cross-sectional area is a different shape from the first end cross-sectional area.

Clause 4. The device of any preceding clause, wherein the center portion includes at least one void within a material of the center portion.

Clause 5. The device of clause 4, wherein the center portion includes a porous material.

Clause 6. The device of any preceding clause, wherein the center portion includes at least one void proximate to an outer surface of the wall.

Clause 7. The device of any preceding clause, wherein the wall of the choke body has a first radiotransparency in a radial sampling direction through the center portion and a second radiotransparency less than the first radiotransparency in a radial transverse direction perpendicular to the radial sampling direction through the center portion.

Clause 8. The device of any preceding clause, wherein the center cross-sectional area has a non-circular shape.

Clause 9. The device of clause 8, wherein the non-circular shape is elongated in a sampling direction.

Clause 10. A system for measuring a composition, the system comprising: a Venturi throat forming a sampling volume; a choke positioned in the Venturi throat wherein the choke reduces a sampling cross-sectional area of the sampling volume to a choke cross-sectional area by a choke ratio of no more than 0.4; a broad-spectrum energy source configured to provide at least a high-energy photon and a low-energy photon to the sampling volume; and a radiation detector located proximate the sampling volume and opposite from the energy source.

Clause 11. The system of clause 10, wherein the broad-spectrum energy source is configured to emit gamma radiation.

Clause 12. The system of clause 10 or 11, wherein at least a portion of the broad-spectrum energy source fluoresces during emission of a photon.

Clause 13. The system of any of clauses 10-12, wherein the choke cross-sectional area is elongated relative to a sampling direction of the broad-spectrum energy source and radiation detector.

Clause 14. The system of any of clauses 10-13, wherein the throat is configured to receive formation fluid from a wellbore.

Clause 15. The system of any of clauses 10-14, wherein a choke major axis of a center cross-sectional area of the choke cross-sectional area is no less than 50% of a throat major axis of a center cross-sectional area of the throat.

Clause 16. The system of any of clauses 10-15, wherein the choke includes PEEK.

Clause 17. The system of any of clauses 10-16, wherein the choke includes a low-absorbing material and a body material different from the low-absorbing material, wherein at least a portion of the low-absorbing material is position between the broad-spectrum energy source and the radiation detector.

Clause 18. A method of measuring a composition, the method comprising: providing a multiphase flow meter (MPFM) having a choke positioned in a sampling volume of the MPFM; flowing a formation fluid through the MPFM, wherein the formation fluid flows through the choke; emitting at least high-energy photons and low-energy photons from an energy source through a center portion of the choke, wherein at least a portion of the high-energy photons and very low-energy photons pass through choke material in a sampling direction; and detecting the portion of the high-energy photons and very low-energy photons after passing through the choke at a detector.

Clause 19. The method of clause 18, wherein emitting the low-energy photons includes energizing a fluorescence source.

Clause 20. The method of clause 18, wherein a sampling path length in which the high-energy photons and very low-energy photons interact with the formation fluid is less than a diameter of the sampling volume of the MPFM.

Clause 21. A kit comprising: a multiphase flow meter (MPFM) including: a throat including a sampling volume; a broad-spectrum energy source configured to provide at least a high-energy photon and a low-energy photon to the sampling volume, and a radiation detector located proximate the sampling volume and opposite from the energy source; and a first choke configured to be positioned in the throat having a first center cross-sectional area less than a sampling volume cross-sectional area of the throat; and a second choke having a second cross-sectional area less than the first center cross-sectional area, wherein the second choke is any choke of clauses 1-17.

It should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein, to the extent such features are not described as being mutually exclusive. Numbers, percentages, ratios, or other values stated herein are intended to include that value, and also other values that are “about”, “substantially”, or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

The terms “approximately,” “about,” and “substantially” as used herein represent an amount close to the stated amount that is within standard manufacturing or process tolerances, or which still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims. The described embodiments are therefore to be considered as illustrative and not restrictive, and the scope of the disclosure is indicated by the appended claims rather than by the foregoing description.

What is claimed is:

1. A device for measuring a composition, the device comprising:
a choke body having a first longitudinal end and a second longitudinal end with a choke volume therein from the first longitudinal end to the second longitudinal end, the choke volume having a center cross-sectional area of a center portion that is no more than a first end cross-sectional area; and
a wall of the choke body that continuous in a circumferential direction and having a first radiotransparency at a first end portion and having a second radiotransparency less than the first radiotransparency in the center portion proximate to the center cross-sectional area.

2. The device of claim 1, wherein the first end portion and the center portion include different materials.

3. The device of claim 1, wherein the center cross-sectional area is a different shape from the first end cross-sectional area.

4. The device of claim 1, wherein the center portion includes at least one void within a material of the center portion.

5. The device of claim 4, wherein the center portion includes a porous material.

6. The device of claim 1, wherein the center portion includes at least one void proximate to an outer surface of the wall.

7. The device of claim 1, wherein the wall of the choke body has a first radiotransparency in a radial sampling direction through the center portion and a second radiotransparency less than the first radiotransparency in a radial transverse direction perpendicular to the radial sampling direction through the center portion.

8. The device of claim 1, wherein the center cross-sectional area has a non-circular shape.

9. The device of claim 8, wherein the non-circular shape is elongated in a sampling direction.

10. A system for measuring a composition, the system comprising:
a Venturi throat forming a sampling volume;
a choke positioned in the sampling volume wherein the choke reduces a sampling cross-sectional area of the sampling volume to a choke cross-sectional area by a choke ratio of no more than 0.4;
a broad-spectrum energy source configured to provide at least a high-energy photon and a low-energy photon to the sampling volume; and
a radiation detector located proximate the sampling volume and opposite from the energy source.

11. The system of claim 10, wherein the broad-spectrum energy source is configured to emit gamma radiation.

12. The system of claim 10, wherein at least a portion of the broad-spectrum energy source fluoresces during emission of a photon.

13. The system of claim 10, wherein the choke cross-sectional area is elongated relative to a sampling direction of the broad-spectrum energy source and radiation detector.

14. The system of claim 10, wherein the throat is configured to receive formation fluid from a wellbore.

15. The system of claim 10, wherein a choke major axis of the choke cross-sectional area is no less than 50% of a throat major axis of a sampling cross-sectional area of the throat.

16. The system of claim 10, wherein the choke includes PEEK.

17. The system of claim 10, wherein the choke includes a low-absorbing material and a body material different from the low-absorbing material, wherein at least a portion of the low-absorbing material is position between the broad-spectrum energy source and the radiation detector.

18. A method of measuring a composition, the method comprising:
providing a multiphase flow meter (MPFM) having a choke positioned in a sampling volume of the MPFM;
flowing a formation fluid through the MPFM, wherein the formation fluid flows through the choke;
emitting at least high-energy photons and very low-energy photons from an energy source through a center portion of the choke, wherein at least a portion of the high-energy photons and very low-energy photons pass through choke material in a sampling direction; and
detecting the portion of the high-energy photons and very low-energy photons after passing through the choke at a detector.

19. The method of claim 18, wherein emitting the very low-energy photons includes energizing a fluorescence source.

20. The method of claim 18, wherein a sampling path length in which the high-energy photons and very low-energy photons interact with the formation fluid is less than a diameter of the sampling volume of the MPFM.
